SEQUENCE LISTING (11 sequences)

SEQ ID NO: 1 - skipped

SEQ ID NO: 2 - protein, 451 aa, synthetic construct
EVQLLESGGGLVQPGGSLRLSCAASGFTFSNAWMSWVRQAPGKGLEWVSSISVGGHRTYYADSVKGRSTISRDNSKNTLYLQMNSLRAEDTAVYYCARIRVGPSGGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 216 aa, synthetic construct
QSVLTQPPSASGTPGQRVTISCSGSNTNIGKNYVSWYQQLPGTAPKLLIYANSNRPSGVPDRFSGSKSGTSASLAISGLRSEDEADYYCASWDASLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 14 aa, synthetic construct
FSNAWMSWVRQAPG

SEQ ID NO: 5 - protein, 19 aa, synthetic construct
SSISVGGHRTYYADSVKGR

SEQ ID NO: 6 - protein, 14 aa, synthetic construct
ARIRVGPSGGAFDY

SEQ ID NO: 7 - protein, 14 aa, synthetic construct
CSGSNTNIGKNYVS

SEQ ID NO: 8 - protein, 7 aa, synthetic construct
ANSNRPS

SEQ ID NO: 9 - protein, 12 aa, synthetic construct
CASWDASLNGWV

SEQ ID NO: 10 - protein, 120 aa, synthetic construct
EVQLLESGGGLVQPGGSLRLSCAASGFTFSNAWMSWVRQAPGKGLEWVSSISVGGHRTYYADSVKGRSTISRDNSKNTLYLQMNSLRAEDTAVYYCARIRVGPSGGAFDYWGQGTLVTVS

SEQ ID NO: 11 - protein, 110 aa, synthetic construct
QSVLTQPPSASGTPGQRVTISCSGSNTNIGKNYVSWYQQLPGTAPKLLIYANSNRPSGVPDRFSGSKSGTSASLAISGLRSEDEADYYCASWDASLNGWVFGGGTKLTVL

(19) United States
(12) Patent Application Publication
(10) Pub. No.: US 2026/0356927 A1
(43) Pub. Date: Nov. 12, 2026

(54) TREATMENT REGIMENS FOR REDUCING RISK OF MAJOR ADVERSE CARDIOVASCULAR EVENT

(71) Applicant: Abcentra, LLC, Los Angeles, CA (US)

(72) Inventor: Christopher John FARINA, Los Angeles, CA (US)

(21) Appl. No.: 19/464,059

(22) Filed: Jan. 29, 2026

Related U.S. Application Data
(60) Provisional application No. 63/751,335, filed on Jan. 30, 2025.

(51) Int. Cl. C07K 16/18; A61K 39/00; A61K 45/06

(52) U.S. Cl. CPC C07K 16/18; A61K 45/06; A61K 2039/54; A61K 2039/545

(57) ABSTRACT

The disclosure provides methods of reducing the risk of a major adverse cardiovascular event (MACE) in a subject in a patient. The method includes administering to the patient orticumab following a phased dosing regimen, wherein the subject is administered orticumab as a loading dose followed by subsequent maintenance doses monthly, bimonthly, or weekly. The anti-oxLDL antibody may be administered to the subject alone or in combination with one or more additional therapeutic agents, such as one or more lipid lowering agents.

### SEQUENCE LISTING

The instant application contains a Sequence Listing which has been submitted electronically in XML format and is hereby incorporated by reference in its entirety. Said XML copy, created on Jan. 21, 2026, is named 51473-031002_Sequence_Listing_1_20_26 and is 10,649 bytes in size.

### BACKGROUND OF THE INVENTION

Cardiovascular diseases (CVDs) are a leading cause of death, affecting an estimated more than 70 million people in the United States alone. CVD events include major adverse cardiac events (MACEs), which include stroke, need for revascularization (e.g., a need to restore blood flow to parts of the heart when that flow is limited or blocked), cardiac arrhythmia, and acute coronary syndrome (ACS), including unstable angina and myocardial infarction. Patients with prior ACS face higher risk of experiencing a MACE and poorer clinical outcomes. 1 in 4 patients who are discharged from a hospital after surviving ACS will experience a MACE within the following 5 years. Therefore, it is desirable to identify methods to reduce the risk of a MACE in a patient, e.g., one who has experienced a prior ACS of subsequently experience a MACE which may result in severe morbidity or death to the patient.

### SUMMARY OF THE INVENTION

In an aspect, the disclosure provides a method for reducing the risk of a MACE in a subject comprising administering to the subject orticumab using a phased dosing schedule comprising: (i) a loading dose comprising 1245 mg of orticumab; and (ii) a maintenance dose comprising 330 mg of orticumab, wherein the maintenance dose is administered to the subject monthly.

In an aspect, the disclosure provides a method for reducing risk of a MACE in a subject comprising administering to the subject orticumab using a phased dosing schedule comprising: (i) a loading dose comprising 1245 mg of orticumab; and (ii) a maintenance dose comprising 165 mg of orticumab, wherein the maintenance dose is administered to the subject every two weeks.

In an aspect, the disclosure provides a method for reducing the risk of a MACE in a subject comprising administering to the subject orticumab using a phased dosing schedule comprising (i) a loading dose comprising 1245 mg of orticumab; and (ii) a maintenance dose comprising 165 mg of orticumab, wherein the maintenance dose is administered to the subject weekly.

In an aspect, the disclosure provides a method for reducing the risk of a MACE in a subject comprising administering to the subject orticumab using a phased dosing schedule comprising (i) a loading dose comprising 1245 mg of orticumab; and (ii) a maintenance dose comprising 82.5 mg of orticumab, wherein the maintenance dose is administered to the subject weekly.

In some embodiments, the loading dose is administered intravenously. In some embodiments, the maintenance dose is administered subcutaneously. In some embodiments, the maintenance dose is administered 1 month after the loading dose is administered to the subject. In some embodiments, the maintenance dose is administered 2 weeks after the loading dose is administered to the subject. In some embodiments, the maintenance dose is administered 1 week after the loading dose is administered to the subject. In some embodiments, the subject is administered orticumab for at least 28 weeks.

In some embodiments, the subject has had a prior acute coronary syndrome (ACS). In some embodiments, the prior ACS comprises unstable angina (UA). In some embodiments, the prior ACS comprises a myocardial infarction (MI). In some embodiments, the MI is ST-segment elevation myocardial infarction (STEMI). In some embodiments, MI is non-ST-segment elevation myocardial infarction (NSTEMI). In some embodiments, the MI has occurred at least 6 months prior to administration of orticumab.

In some embodiments, the subject has an FAI Percentile of ≥70. In some embodiments, the subject has an FAI Percentile of ≥50 and <70 (e.g., between 50 and 68, 50 and 65, 50 and 62, 50 and 60, 50 and 57, 50 and 55, 50 and 52, 52 and 69, 55 and 69, 57 and 69, 60 and 69, 62 and 69, 65 and 69, 67 and 69, 55 and 65, or 52 and 62).

In some embodiments, the subject's relative risk of a MACE is reduced by at least 20%. In some embodiments, the subject's relative risk of a MACE is reduced by at between 20% and 99% (e.g., between 20% and 90%, 20% and 80%, 20% and 70%, 20% and 60%, 20% and 50%, 20% and 40%, 20% and 30%, 30% and 99%, 40% and 99%, 50% and 99%, 60% and 99%, 70% and 99%, 80% and 99%, 40% and 70%, and 30% and 60%).

In some embodiments, the MACE is an MI. In some embodiments, the MACE is a stroke. In some embodiments, the MACE is resuscitated cardiovascular death.

In some embodiments, the method further comprises administering a lipid-lowering agent to the subject. In some embodiments, the subject was previously on a lipid-lowering agent. In some embodiments, the lipid-lowering agent comprises an antibody, an siRNA, an antisense oligonucleotide, a small molecule, an omega-3 fatty acid, a bile acid sequestrant, or a peptide.

In some embodiments, the small molecule is a statin. In some embodiments, the statin is lovastatin, pravastatin, simvastatin, fluvastatin, atorvastatin, rosuvastatin, or pitavastatin. In some embodiments, the small molecule is ezetimibe. In some embodiments, the small molecule is a fibrate. In some embodiments, the fibrate is fenofibrate, bezafibrate, gemfibrozil, or pemafibrate. In some embodiments, the lipid-lowering agent is a bile acid sequestrant. In some embodiments, the bile acid sequestrant is cholestyramine, colesevelam or colestipol. In some embodiments, the small molecule is bempedoic acid or lomitapide. In some embodiments, the lipid-lowering agent is an omega-3 fatty acid. In some embodiments, the omega-3 fatty acid is docosahexaenoic acid, eicosapentaenoic acid, or docosapentaenoic acid.

In some embodiments, the lipid-lowering agent is an antibody. In some embodiments, the antibody is a PCSK9 antibody. In some embodiments, the PCSK9 antibody is evolocumab or alirocumab. In some embodiments, the antibody is evinacumab.

In some embodiments, the lipid-lowering agent is an siRNA. In some embodiments, the siRNA is a PCSK9 siRNA. In some embodiments, the PCSK9 siRNA is inclisiran. In some embodiments, the siRNA is olpasiran. In some embodiments, the lipid-lowering agent is an antisense oligonucleotide. In some embodiments, the antisense oligonucleotide is mipomersen, volanesorsen, pelacarsen, or olezarsen. In some embodiments, the peptide is NNC0385-0434A.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 is a scheme showing the dosing schedule for administration of orticumab to a subject.

FIGS. 2A and 2B show the simulated pharmacokinetic (PK) profiles of various dosing schedules. FIG. 2A shows administration of a 1245 mg loading dose of orticumab followed by administration of maintenance doses of 1245 mg monthly in comparison to administration of a loading dose of 1245 mg of orticumab followed administration of maintenance doses of 330 mg of orticumab administered monthly. FIG. 2B shows the simulated PK profile of three dosing schedules comprising a loading dose of orticumab comprising 1245 mg followed by subcutaneous maintenance doses of either 330 mg orticumab administered monthly, 165 mg orticumab administered every two weeks, or 82.5 mg orticumab administered weekly.

FIG. 3 shows the simulated PK profile of the dosing schedule of a loading dose of 1245 mg intravenous bolus of orticumab followed by a subcutaneous weekly maintenance dose of 165 mg of orticumab, showing cumulative exposure of orticumab.

### DEFINITIONS

Terms used in the claims and specification are defined as set forth below unless otherwise specified.

As used in the specification and the appended claims, the singular forms “a,” “an,” and “the” include plural referents unless the context clearly dictates otherwise.

As used herein, “about” means plus or minus 10% of the particular value.

The term “acute coronary syndrome,” as used herein, encompasses any group of clinical symptoms compatible with acute myocardial ischemia. Acute myocardial ischemia causes chest pain due to insufficient blood supply to the heart muscle that results from coronary artery disease, which is also known as coronary heart disease. Acute myocardial ischemia includes unstable angina (UA) and (acute) myocardial infarction (AMI or MI), in which the heart muscle is damaged. UA, which may also be referred to as crescendo angina, refers to angina pectoris that changes or worsens. AMI refers to both non-ST segment elevation myocardial infarction (NSTEMI) and ST segment elevation myocardial infarction (STEMI). These two types of MI may be determined using an electrocardiogram (EKG).

“Administering” and “administer,” as used herein, refer to any route for delivering a pharmaceutical composition to a patient. Routes of delivery may include parenteral routes and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

As used herein, the terms “antibody” and “antibodies” are meant in a broad sense and includes immunoglobulin molecules including polyclonal antibodies, monoclonal antibodies including murine, human, human-adapted, humanized and chimeric monoclonal antibodies, antibody fragments, bispecific or multispecific antibodies, dimeric, tetrameric or multimeric antibodies, and single chain antibodies.

Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (κ) and lambda (λ), based on the amino acid sequences of their constant domains.

As used herein, the term “reduce the risk of a major cardiovascular event” refers to reducing the likelihood that a patient undergoes a major adverse cardiovascular event (MACE). A reduced risk of MACE is assessed for a given patient population (e.g., patients who have experienced a prior acute coronary syndrome (ACS)). The reduction in risk for MACE is understood by comparing rates for a MACE in patients having prior ACS being treated by the methods described herein with the rates of MACE in patients having prior who are not treated by the methods described herein. A therapeutically or prophylactically significant reduction in occurrence of a MACE is, e.g., at least 5%, at least 10%, at least 20%, at least 30%, at least 40%, at least 50%, at least 60%, at least 70%, at least 80%, at least 90%, at least 100%, or more in a measured parameter as compared to a control or non-treated patient or the state of the patient prior to administering the compositions described herein.

The term “in combination with” as used herein means that two or more therapeutics can be administered to a patient together in a mixture, concurrently as single agents, or sequentially as single agents in any order.

As used herein, the term “major cardiovascular event” or “MACE” encompasses stroke, need for revascularization (e.g., a need to restore blood flow to parts of the heart when that flow is limited or blocked), cardiac arrhythmia, and ACS, including UA and MI. Potential outcomes of MACEs include fatal MI, fatal stroke, nonfatal MI, nonfatal stroke, nonfatal UA, and heart failure requiring hospitalization.

As used herein, the term “orticumab” refers a fully human recombinant IgG1 monoclonal antibody (molecular weight, approximately 144 kDa) having an HCDR1, HCDR2, HCDR3 of orticumab are set forth in SEQ ID NOs: 4, 5 and 6, respectively; and LCDR1, LCDR2, and LCDR3 are set forth in SEQ ID NOs: 7, 8 and 9, respectively. Orticumab further comprises a heavy chain amino acid sequence that is at least 90% (e.g., at least 91%, 92%, 93%, 94%, 95%, 96%, 97%, 98%, 99%, or 100%) identical to the amino acid sequence of SEQ ID NO: 2, and light chain amino acid sequence that is at least 90% (e.g., at least 91%, 92%, 93%, 94%, 95%, 96%, 97%, 98%, 99%, or 100%) identical of SEQ ID NO: 3. The heavy chain of orticumab comprises a variable heavy region (V_{H}) amino acid sequence that is at least 90% (e.g., at least 91%, 92%, 93%, 94%, 95%, 96%, 97%, 98%, 99%, or 100%) identical to the amino acid sequence of SEQ ID NO: 10, and the light chain of orticumab comprises a variable light region (V_{L}) amino acid sequence that is at least 90% (e.g., at least 91%, 92%, 93%, 94%, 95%, 96%, 97%, 98%, 99%, or 100%) identical of SEQ ID NO: 11.

As used herein, the terms “pericoronary vascular adipose tissue,” “PVAT,” and “coronary PVAT attenuation” describe the average coronary computed tomography angiography (CCTA) attenuation in Hounsfield units (HU) of the adipose tissue inside the designated volume of interest, while adipose tissue may be defined as all voxels having attenuation between −190 and −30 HU.

As used herein, the term “perivascular FAI” and “FAI” is defined as the weighted mean attenuation of all adipose tissue-containing voxels (−190 HU to −30 HU) lying within a radial distance from the outer vessel wall equal to the diameter of the relevant vessel around the coronary vessels. To avoid the effects of the aortic wall, the most proximal 10 mm segment may be excluded as well as the proximal 10-50 mm of the coronary vessel in most of the studies.

“Subject,” “individual,” “animal,” “patient,” and “mammal” refer to any subject, particularly a mammalian subject, for whom diagnosis, prognosis, or therapy is desired. Mammalian subjects include, but are not limited to, humans, domestic animals, farm animals, zoo animals, sport animals, pet animals such as guinea pigs, rabbits, rats, mice, cattle; primates such as apes, monkeys, orangutans, and chimpanzees; canids such as dogs and wolves; felids such as cats, lions, and tigers; equids such as horses, donkeys, and zebras; food animals such as cows, pigs, and sheep; and ungulates such as deer and giraffes. In certain embodiments, the mammal is a human subject.

As used herein, the terms “treat,” “treatment,” “treating,” and “amelioration,” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease-state is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results or lower the chances of the individual developing the condition even if the treatment is ultimately unsuccessful. Those in need of treatment include those already with the condition as well as those prone to have the condition or those in whom the condition is to be prevented.

### DETAILED DESCRIPTION

The present disclosure features compositions and methods of reducing the risk of a major adverse cardiovascular event (MACE) in a patient. The method includes administering to the patient orticumab following a phased dosing regimen, wherein the subject is administered orticumab as a loading dose followed by subsequent maintenance doses monthly, bimonthly, or weekly. Orticumab may be incorporated into a pharmaceutical composition, which can be administered to a patient according to the described dosing regimen alone or in combination with another therapeutical agent, such as a lipid-lowering agent to reduce a patient's risk of experiencing a MACE.

MACE encompasses stroke, need for revascularization (e.g., a need to restore blood flow to parts of the heart when that flow is limited or blocked), cardiac arrhythmia, and acute coronary syndrome (ACS), including UA and MI. Potential outcomes of a patient experiencing a MACE include cardiovascular death as a result of fatal MI and fatal stroke, nonfatal MI, nonfatal stroke, nonfatal UA, or heart failure requiring hospitalization. The methods described herein reduce the risk of a MACE in a patient who has had a prior ACS, where the risk of a MACE refers to the potential likelihood that a patient experiences a MACE. A reduced risk of MACE is assessed for a given patient population (e.g., patients who have experienced a prior ACS) of a particular severity comparing rates for a MACE in patients being treated by a method of the invention alone or in combination with a lipid-lowering agent to patients relying solely on a lipid-lowering agent. As a result of treatment by the methods described herein, the patient may have a reduced risk of having a MACE. The risk may be reduced by at least 5%, at least 10%, at least 20%, at least 30%, at least 40%, at least 50%, at least 60%, at least 70%, at least 80%, at least 90%, at least 100%, or more in comparison to a patient who has not been administered orticumab after an ACS. For example, the patient who is administered orticumab as described herein may reduce their relative risk of a MACE by 5% to 100% (e.g., 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 5% to 15%, 5% to 10%, 10% to 15%, 10% to 20%, 10% to 25%, 10% to 30%, 10% to 35%, 10% to 40%, 10% to 45%, 10% to 50%, 10% to 55%, 10% to 60%, 10% to 65%, 10% to 70%, 10% to 75%, 10% to 80%, 10% to 85%, 10% to 90%, 10% to 95%, 10% to 100%, 20% to 50%, or 50% to 80%). The patient's relative risk of a MACE may be reduced by at least 20%. In some embodiments, the patient's relative risk of a MACE is reduced by 20% to 99% (e.g., 20% to 90%, 20% to 70%, 20% to 50%, 20% to 30%, 30% to 99%, 50% to 99%, 70% to 99%, 90% to 99%, 30% to 50%, 40% to 70%, 30% to 60%, or 50% to 80%). Embodiments of the present methods can also be used to assess the patient's risk of experiencing an adverse cardiac event within the ensuing 30 days, 60 days, 90 days, five years, four years, three years, two years, or one year. In another embodiment, the present methods are used to determine if a patient presenting with chest pain is at risk of experiencing a heart attack or other MACE, such as a near term MI, reinfarction, stroke, the need for revascularization (e.g., a need to restore blood flow to parts of the heart when that flow is limited or blocked), or death. As used in this context, the term “near term” means within one year. Thus, patients who are at near term risk may be at risk of experiencing a MACE within the following day, 3 months, or 6 months after presenting with chest pain.

### Methods of Administration

Orticumab may be administered using the dosing schedules described below to reduce the patient's risk of a MACE. In some embodiments, orticumab is administered to the subject that has had an ACS prior to administration of orticumab. Orticumab may be administered to the patient in order to reduce the patient's risk of MACE either alone or in combination with one or more therapeutic agents. For example, orticumab may be administered to the patient with one or more lipid lowering agents. In some embodiments the patient is assessed using Coronary Computed Tomography Angiography (CCTA). For example, the CCTA may be used to assess the pericoronary vascular adipose tissue or perivascular Fat Attenuation Index in the patient.

The subject's risk of a MACE may be reduced by administering to the subject orticumab using a phased dosing schedule. Three phased dosing schedules are proposed to achieve target concentration ranges, schedules start with a 1245 mg orticumab loading dose followed by: (i) 330 mg of orticumab administered monthly subcutaneously; (ii) 165 mg of orticumab administered bimonthly, subcutaneously; (iii) 165 mg of orticumab administered weekly, subcutaneously; or 82.5 mg of orticumab administered weekly, subcutaneously.

The loading dose of the dosing schedule may be administered to the subject intravenously. The maintenance dose of the dosing schedule may be administered subcutaneously. The maintenance dose may be administered to the subject about 1 week (e.g., 5 days, 6 days, 7 days, or 8 days) after the loading dose is administered to the subject. Orticumab may be administered to the subject for at least 12 weeks. Orticumab may be administered to the subject for at least 24 weeks. Orticumab may be administered to the subject for at least 28 weeks. Orticumab may be administered to the subject for at least 52 weeks. The maintenance dose may be administered to the subject for at least 10 weeks. The maintenance dose may be administered to the subject for at least 23 weeks. The maintenance dose may be administered to the subject for at least 27 weeks.

### Combination Therapies

Orticumab may be administered to the patient in combination with one or more additional therapeutic agents. For example, orticumab may be administered in combination with a lipid-lowering agent. In some embodiments, the patient may have been administered a lipid-lowering agent prior to experiencing ACS. In some embodiments, the patient is administered orticumab prior to administration of a lipid lowering agent. In some embodiments, the patient is administered lipid lowering agent prior to the administration of orticumab. In some embodiments, the patient is administered orticumab and the lipid lowering agent concurrently. In some embodiments, the lipid lowering agent is administered using the same dosing regimen as orticumab. In some embodiments, the lipid lowering agent is administered to the subject using a dosing regimen that is different from the dosing regimen of orticumab.

The lipid-lowering agent may be an antibody, an siRNA, an antisense oligonucleotide, a small molecule, an omega-3 fatty acid, a bile acid sequestrant, or a peptide.

The lipid-lowering agent may be a small molecule. In some embodiments, the lipid-lowering agent is a statin. For example, the statin may be lovastatin, pravastatin, simvastatin, fluvastatin, atorvastatin, rosuvastatin, or pitavastatin, or a combination thereof. Statins inhibit 2-hydroxyl-methyl-glutaryl coenzyme A (HMG-COA) reductase, which is the rate-limiting step in cholesterol synthesis. As a result, there may be a reduced intracellular cholesterol concentration and subsequent upregulation of LDL receptors which encourages the removal of low-density lipoprotein cholesterol (LDL-C) from the circulation. The resulting reduction in LDL-C as a result of administering a statin to the patient may be dose dependent. The statin may also reduce the level of triglycerides in a patient upon administration. While statins are generally safe and generally tolerated by most patients, higher doses that achieve greater LDL-C reduction may cause more side effects such as gastrointestinal disturbances and myopathy. Increase in liver enzymes, particularly alanine and aspartate transaminases may occur in some patients who are administered a statin therapy and may normalize on reducing the dose or stopping the statin. A baseline measurement of liver function test may be performed prior to beginning the statin therapy. Statin therapy may be discontinued if transaminases are found to be well over a reference limit.

In some embodiments, the small molecule is ezetimibe. Ezetimibe acts by inhibiting cholesterol absorption through inhibition of the duodenal Niemann-Pick C1-like protein. Ezetimibe may be administered to the patient alone or in combination with one or more lipid-lowering agents. For example, ezetimibe may be administered to a patient in combination with a statin.

In some embodiments, the small molecule is a fibrate. For example, the fibrate may be fenofibrate, bezafibrate, gemfibrozil, or pemafibrate. Fibrates reduce concentrations of triglycerides. Treatment with fibrates may cause side effects including gastrointestinal pain, skin rashes, myopathy, and liver enzyme elevation. The fibrate may be administered to the patient alone or in combination with one or more lipid-lowering agents. For example, the fibrate may be administered to a patient in combination with a statin. In some embodiments, the small molecule is bempedoic acid or lomitapide.

In some embodiments, the lipid-lowering agent is a bile acid sequestrant. For example, the bile acid sequestrant may be cholestyramine, colesevelam, or colestipol. Bile acid sequestrants bind to intestinal cholesterol and thereby inhibit the absorption of cholesterol in the small intestine. As a result, intestinal cholesterol may be effectively removed from the enterohepatic cholesterol circulation. Treatment with bile sequestrants is associated with a significant reduction in LDL-Cs. The bile acid sequestrant may be administered to the patient alone or in combination with one or more lipid-lowering agents. For example, the bile acid sequestrant may be administered to a patient in combination with a statin.

In some embodiments, the lipid-lowering agent is an omega-3 fatty acid. For example, the omega-3 fatty acid may be docosahexaenoic acid (DHA), eicosapentaenoic acid (EPA), or docosapentaenoic acid (DPA). The omega-3 fatty acid may be administered to the patient alone or in combination with one or more additional lipid-lowering agents. For example, the patient may be administered a statin and an omega-3 fatty acid. The omega-3 fatty acid may reduce triglycerides in the patient in a dose-dependent manner.

In some embodiments, the lipid-lowering agent may be an antibody. For example, the antibody may be evinacumab. Evinacumab inhibits angiopoietin-like 3 (ANGPTL3), which results in decreased blood lipid levels. In some embodiments, the antibody may be a PCSK9 antibody, such as evolocumab or alirocumab. PCSK9 antibodies inhibit PCSK9, thereby reducing the concentration of PCSK9 plasma levels. As a result, LDL-C receptor expression is increased, and LDL-C levels are reduced. PCSK9 inhibitors also lower triglycerides and lipoprotein(a). Orticumab may be administered to the patient alone or in combination with one or more lipid-lowering agents. For example, orticumab may be administered to a patient in combination with a statin.

In some embodiments, the lipid-lowering agent may be an siRNA. For example, the siRNA may be olpasiran. Olpasiran acts by reducing Lp(a) levels. In some embodiments, the siRNA may be a PCSK9 siRNA, such as inclisiran. The PCSK9 siRNA may act by inhibiting PCSK9 biosynthesis. The underlying method of action is similar to evolocumab and alirocumab. The siRNA permits infrequent subcutaneous administration with fewer injection site reactions. The siRNA may be administered to the patient alone or in combination with one or more lipid-lowering agents. For example, the siRNA may be administered to a patient in combination with a statin.

In some embodiments, the lipid-lowering agent may be an antisense oligonucleotide. For example, the antisense oligonucleotide may be mipomersen, volanesorsen, pelacarsen, or olezarsen. The antisense oligonucleotide may be administered to the patient alone or in combination with one or more lipid-lowering agents. For example, the antisense oligonucleotide may be administered to a patient in combination with a statin.

In some embodiments, the lipid-lowering agent may be a peptide. For example, the peptide may be NNC0385-0434A. NNC0385-0434A is a PCSK9 inhibitor. The peptide may be administered to the patient alone or in combination with one or more lipid-lowering agents. For example, the peptide may be administered to a patient in combination with a statin.

### Assessment of Treatment

The subject may be assessed before treatment with the dosing schedule described herein and after treatment with the dosing schedule described herein by way of an FAI score or FAI percentile score. The subject may experience a reduction of FAI score or FAI score percentile as a result of administration of orticumab. A subject may be evaluated for their FAI score and/or FAI score percentile prior to administration of orticumab described herein using computed tomography (CT) to scan a blood vessel of the subject. For example, the computed tomography may be coronary computed tomography angiography (CCTA). The scan may be used to characterize the severity of the inflammation of the blood vessel. For example, the CT scan may be used to measure the inflammation of the vessel wall by measuring the ratio of the thickness of the vascular wall to the thickness of the perivascular adipose tissue (PVAT) within the blood vessel. Accordingly, the subject's fat attenuation index (FAI), FAI score, and/or FAI percentile may be assessed prior to treatment and monitored after treatment with orticumab has progressed. The subject may have an FAI percentile score prior to treatment with orticumab of ≥70. For example, the FAI percentile score may be 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 10. The subject may have an FAI percentile score between 50 and 70 before treatment with orticumab. For example, the subject may have an FAI percentile score of 50, 51, 52, 53, 54, 55, 56, 57, 58, 59, 60, 61, 62, 63, 64, 65, 66, 67, 68, 69, and 70 prior to administration of orticumab.

The FAI is an unadjusted, visual representation of the extent of coronary inflammation in the blood vessels of a subject. To determine the FAI, the coronary inflammation is assessed in the three main epicardial coronary arteries including the right coronary artery (RCA), the left anterior descending artery (LAD), and the left circumflex artery (LCX). The FAI measures changes in PVAT composition which are driven by inflammatory signals coming from the inflamed coronary artery via analyzing the 3D gradients of perivascular tissue attenuation, which then is adjusted for technical factors as well as anatomical and biological factors pertaining to the subject. The FAI is the weighted mean attenuation of all adipose tissue-containing voxels lying within a radial distance from the outer wall of the blood vessel equal to the diameter of the relevant vessel around the coronary vessels. Specifically, PVAT is defined as adipose tissue located within a radial distance equal to the diameter of the respective vessel extending from the outer vessel wall. This is based on a biological definition of PVAT derived from adipose tissue biopsies from the perivascular area which demonstrates a different adipose tissue phenotype: having smaller adipocytes as well as lower expression of adipogenic genes and less lipophilic/greater aqueous phase close to the vessel compared with adipose tissue 2 cm away from the vascular wall. Additionally, this method of measuring may be employed as the mean attenuation of PVAT has been shown to be independent of lumen attenuation, thus avoiding a partial volume effect.

FAI values may be calculated to avoid the effects of the aortic wall, such as, the most proximal 10 mm segment as well as the proximal 10-50 mm of the coronary vessel, which are commonly excluded from calculations. As described in Sagris et al., European Heart Journal—Cardiovascular Imaging, 23 (12), December 2022, 526-553, which is herein incorporated by reference in its entirety, the proximal 40 mm segment of the LAD, the LCX, and the RCA may be manually traced. The analysis may be performed by segmenting the perivascular area into 20 concentric cylindrical layers of 1 mm thickness each. Voxel attenuation histograms are plotted, and the mean attenuation of all voxels characterized as adipose tissue within this volume are defined as FAI_{PVAT}. Next, the respective FAI index is calculated for adipose tissue in each of the 20 concentric cylindrical layers and is plotted against the radial distance from the outer vessel wall. On the other hand, FAI_{non-PVAT} is defined as the FAI value of adipose tissue in the most distal cylindrical layer (2 cm away from the vascular wall). In order to describe the change in adipose tissue attenuation between PVAT and non-PVAT, the volumetric perivascular characterization index (VPCI) is created and defined as the % change from FAI_{PVAT} to FAI_{non-PVAT} [VPCI=100×(FAI_{PVAT}-FAI_{non-PVAT})/|FAI_{PVAT}|]. VPCI correlates with the presence of “soft atherosclerotic plaques,” as defined by using the standard plaque analysis methodology.

Further methods of determining the FAI for a subject using computed tomography are also disclosed in U.S. Pat. No. 10,695,023, U.S. Patent Publication No. US 2022/0139005, International Patent Application WO 2016/024128; Oikonomou et al. Lancet, 10151 (392), September 2018, 929-939; Antonopoulos et al., Sci. Transl. Med., 9, 2017; which are herein incorporated by reference in their entirety.

The FAI score quantifies the changes in the perivascular adipose tissue triggered by inflammatory signals derived from the vascular wall and provides a quantitative metric of coronary inflammation with major prognostic value. The FAI score is an individualized quantification of coronary inflammation in the three main epicardial coronary arteries, adjusted for age and gender and can be viewed as a measure of disease activity. The FAI Score standardizes the FAI for technical, anatomical, and biological factors before its introduction in the clinic as a potential risk stratification. The FAI Score utilizes the power of artificial intelligence to standardize FAI values, as described above, from coronary arteries for a clinical metric of coronary inflammation. The score was initially standardized using 3912 individuals, with 2040 from the United States and 1872 from Europe, having a mean age of 55.7, 41.1% of whom were women; all underwent clinically indicated coronary computed tomography angiography (CCTA) as described in Oikonomou et al. (Lancet, 10151 (392), September 2018, 929-939), which is herein incorporated by reference in its entirety. FAI Score is measured at baseline in the proximal RCA, LAD, and LCX.

As described by Oikonomou et al. (European Journal of Preventive Cardiology, 28(1), May 2021), which is herein incorporated by reference in its entirety, a dissimilarity index may be computed to classify individuals based on pre-randomization characteristics according to the Gower distance, which is a metric of dissimilarity between two subjects based on mixed numeric and non-numeric data. For continuous variables, Gower distance represents the absolute value of the difference between a pair of individuals divided by the range across all individuals. For categorical variables, the method assigns “1” if the values are identical and “0” if they are not. A Gower distance may ultimately be calculated as the mean of these terms. Alternatively, the dissimilarity index can be computed based on cosine similarity or other similarity measures. For each subject, a topological neighborhood may be identified as the 5% most phenotypically similar participants based on Gower's distance. In sensitivity analyses, random neighborhood sizes between 2.5% and 10% may be iteratively evaluated, assessing the correlation of effect estimates in these iterations with those derived from the 5% neighborhood size.

Within each subject-centered neighborhood, the association of undergoing anatomical vs. functional imaging is assessed with major adverse cardiac event in age- and sex-adjusted Cox regression models, which thus provides individualized risk estimates based on each subject's unique neighborhood. The natural logarithmic transformations of the hazard ratio (HR) from the Cox models comparing anatomical and functional testing for each subject's topological neighborhood represent their individualized effect estimate. Negative log-HRs favor anatomical testing, whereas positive values favor functional imaging. Furthermore, since an unbiased personalized effect estimate is contingent upon the similarity of individuals in their topological neighborhoods, a measure of neighborhood homogeneity may be created. This represents the square of 1 minus the average pairwise distance between the index subject and each one of their neighbors, with higher values reflecting a neighborhood of phenotypically more similar subjects.

Methods of determining FAI scores are disclosed in US 2022/0336048 and Oikonomou et al., Cardiovascular Research 117(13), November 2021, 2677-2690, which are herein incorporated by reference in their entirety.

The percentile curves associated with the FAI Score are estimates of how the score is distributed among a group of subjects who have undergone a clinically indicated CT. These curves provide a way to compare an individual's FAI Score to the others in the same age and gender group. Subjects in the upper percentile curves have an increased risk of cardiovascular disease, regardless of the presence of traditional risk factors or established atherosclerotic changes

FAI Score percentiles emerged from FAI Score phenotyping of three main coronary areas as estimates of the distribution. Phenotyping of the FAI Score shows that there is a stepwise association between higher FAI Score percentiles and higher prospective risk of cardiac mortality in both univariate and multivariable Cox-regression. Nomograms and percentile curves are calculated for FAI Score around each one of the coronary vessels in a pooled analysis.

### Anti-Ox-LDL Antibodies

The disclosure provides methods of reducing the risk of a MACE in a patient, wherein the patient may have previously had an ACS by administering to the patient orticumab. Orticumab is an antibody capable of binding to the ox-LDL ApoB100.

The HCDR1, HCDR2, HCDR3 of orticumab are set forth in SEQ ID NOs: 4, 5 and 6, respectively; and LCDR1, LCDR2, and LCDR3 are set forth in SEQ ID NOs: 7, 8 and 9, respectively.

HCDR1:
(SEQ ID NO: 4)
FSNAWMSWVRQAPG.
HCDR2:
(SEQ ID NO: 5)
SSISVGGHRTYYADSVKGR.
HCDR3:
(SEQ ID NO: 6)
ARIRVGPSGGAFDY.
LCDR1:
(SEQ ID NO: 7)
CSGSNTNIGKNYVS.
LCDR2:
(SEQ ID NO: 8)
ANSNRPS
LCDR3:
(SEQ ID NO: 9)
CASWDASLNGWV.

Orticumab contains a variable heavy region (V_{H}) amino acid sequence that is at least 90% (e.g., at least 91%, 92%, 93%, 94%, 95%, 96%, 97%, 98%, 99%, or 100%) identical to the amino acid sequence of SEQ ID NO: 10, and a variable light region (V_{L}) amino acid sequence that is at least 90% (e.g., at least 91%, 92%, 93%, 94%, 95%, 96%, 97%, 98%, 99%, or 100%) identical of SEQ ID NO: 11. In one embodiment, orticumab contains a variable heavy region (V_{H}) amino acid sequence of SEQ ID NO: 10, and a variable light region (V_{L}) amino acid sequence of SEQ ID NO: 11. Orticumab contains a heavy chain amino acid sequence that is at least 90% (e.g., at least 91%, 92%, 93%, 94%, 95%, 96%, 97%, 98%, 99%, or 100%) identical to the amino acid sequence of SEQ ID NO: 2, and light chain amino acid sequence that is at least 90% (e.g., at least 91%, 92%, 93%, 94%, 95%, 96%, 97%, 98%, 99%, or 100%) identical of SEQ ID NO: 3. In one embodiment, orticumab contains a heavy chain amino acid sequence of SEQ ID NO: 2, and a light chain amino acid sequence of SEQ ID NO: 3. In some embodiments, orticumab may contain between one and five (e.g., one, two, three, four, or five) amino acid substitutions in the heavy chain amino acid sequence of SEQ ID NO: 2, which includes a V_{H}, a hinge region, and three constant heavy chain regions. In some embodiments, orticumab comprises a heavy chain amino acid sequence of SEQ ID NO: 2, which includes a V_{H}, a hinge region, and three constant heavy chain regions. In some embodiments, orticumab may contain between one and five (e.g., one, two, three, four, or five) amino acid substitutions in the light chain amino acid sequence of SEQ ID NO: 3, including a V_{L} and constant light chain region. In some embodiments, orticumab comprises a light chain amino acid sequence of SEQ ID NO: 3, including a V_{L} and constant light chain region. In some embodiments, orticumab comprises a heavy chain of SEQ ID NO: 2, wherein the terminal Lys reside is optionally absent. The amino acid sequence of orticumab is also described in WO 2009/083225 and U.S. Patent Application Publication No. US 2011/0014203, which are incorporated by reference herein in their entireties.

(SEQ ID NO: 2)
EVQLLESGGGLVQPGGSLRLSCAASGFTFSNAWMSWVRQAPGKGLEWVSS
ISVGGHRTYYADSVKGRSTISRDNSKNTLYLQMNSLRAEDTAVYYCARIR
VGPSGGAFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K

(SEQ ID NO: 3)
QSVLTQPPSASGTPGQRVTISCSGSNTNIGKNYVSWYQQLPGTAPKLLIY
ANSNRPSGVPDRFSGSKSGTSASLAISGLRSEDEADYYCASWDASLNGWV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

(SEQ ID NO: 10)
EVQLLESGGGLVQPGGSLRLSCAASGFTFSNAWMSWVRQAPGKGLEWVS
SISVGGHRTYYADSVKGRSTISRDNSKNTLYLQMNSLRAEDTAVYYCAR
IRVGPSGGAFDYWGQGTLVTVS,
(SEQ ID NO: 11)
QSVLTQPPSASGTPGQRVTISCSGSNTNIGKNYVSWYQQLPGTAPKLLI
YANSNRPSGVPDRFSGSKSGTSASLAISGLRSEDEADYYCASWDASLNG
WVFGGGTKLTVL.

### Pharmaceutical Compositions

In various embodiments, the present disclosure provides a pharmaceutical composition for use in the method of reducing the risk of a patient having a MACE described herein. The pharmaceutical composition includes orticumab and a pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof. Generally, each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions may be delivered in according to the dosing schedules described herein.

In various embodiments, orticumab can be formulated for delivery via any route of administration. For example, the methods include administration via an aerosol, nasal, oral, transmucosal, transdermal, parenteral or enteral route. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Administration of Orticumab Via Different Loading Schedules

Simulations to assess the resulting pharmacokinetic (PK) profile of various dosing regimens of orticumab were performed to identify an effective and tolerable dose of orticumab to administer to subjects in order to reduce the subject's risk of a MACE.

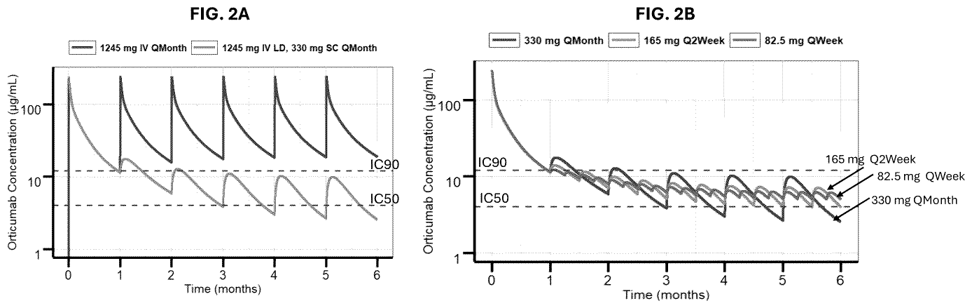

Comparison of a different dosing regimens were simulated with the aim to maintain an average blood concentration of orticumab that maintained exposure between 4 μg/mL (calculated IC50) and 12 μg/mL (calculated IC90). Simulated PK exposure was accordingly performed for intravenous administration of a loading dose of 1245 mg of orticumab followed by: (i) a maintenance dose of 1245 mg of orticumab administered monthly, intravenously (high dose) and (ii) a maintenance dose of 330 mg of orticumab administered monthly, subcutaneously (FIG. 2A). The regimen having a maintenance dose of 1245 mg of orticumab had troughs consistently above 12 μg/mL while the regimen having a maintenance dose of 330 mg of orticumab had peaks around 12 μg/mL and troughs descending slightly below 4 μg/mL.

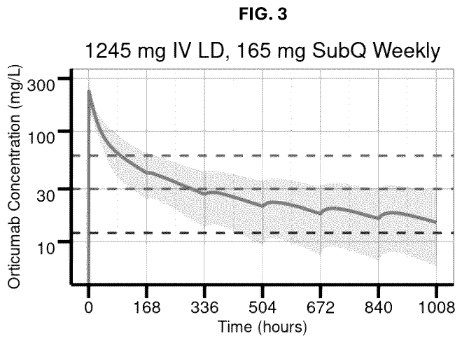

Further looking into various dosing schedules, the aim of this study was to use simulations to maintain an average blood concentration of orticumab that maintained exposure between 4 μg/mL (IC5) and 12 μg/mL (IC90). Simulated PK exposure was accordingly performed for intravenous administration of a loading dose of 1245 mg of orticumab followed by maintenance doses of: (i) 330 mg of orticumab administered monthly subcutaneously; (ii) 165 mg of orticumab administered bimonthly, subcutaneously; (iii) 165 mg of orticumab administered weekly, subcutaneously; or (iv) 82.5 mg of orticumab administered weekly, subcutaneously (FIGS. 2 and 3). For subjects receiving an initial loading dose of 1245 mg orticumab followed by monthly maintenance doses of 330 mg of orticumab, at the 24 week timepoint, orticumab concentration decreases to below 4 μg/mL (FIG. 2B). For subjects receiving an initial loading dose of 1245 mg orticumab followed by biweekly maintenance doses of 165 mg of orticumab, from 4 week to 24 week timepoint, orticumab concentration has troughs at 4 μg/mL and peaks at 12 μg/mL, achieving steady state exposure of about 7.4 μg/mL (FIG. 2B). For subjects receiving an initial loading dose of 1245 mg followed by weekly maintenance doses of 165 mg of orticumab, from 336 hour to 1008 hours, orticumab concentration had troughs at 9.1 μg/mL and steady state max concentration of 11.2 μg/mL (FIG. 3). For subjects receiving an initial loading dose of 1245 mg followed by weekly maintenance doses of 82.5 mg of orticumab, from 4 week to 24 week timepoint, orticumab concentration stays between 4 μg/mL and 12 μg/mL (FIG. 2B).

The PK modeling was developed using the nonlinear mixed-effects modeling approach in Pumas (version 2.4, Pumas AI, Baltimore MD, https://pumas.ai/). The First-Order Conditional Estimation (FOCE) without eta-epsilon interaction method was used to fit the PK data and estimate the typical values of PK parameters (structural model), the between-subject variability (BSV) for typical PK parameters (random effects model), as well as the within-subject variability (residual error model) in Orticumab concentrations. Uncertainty estimates for population parameters were determined using the infer function of Pumas software, which evaluates the asymptotic variance-covariance matrix obtained from the robust (sandwich) estimator. Uncertainty estimates for population parameters were determined using the infer function of Pumas software.

### Example 2: Multicenter, Double-Blind, Randomized, Placebo-Controlled Study of Orticumab in Participants with Prior Myocardial Infarction Who have Elevated Coronary Inflammation Based on Fat Attenuation Index (FAI) Score Assessed by Coronary Computed Tomography Angiography (CCTA)

### Rationale for Study Population

OxLDL accumulates in atherosclerotic plaques and contributes to a highly inflammatory milieu in these lesions.

Acute coronary syndrome (ACS) is associated with significantly increased oxidative stress and inflammation. Inflammation after an acute myocardial infarction event, including the level of oxLDL, spike. This inflammation drives risk of a recurrent event. Despite contemporary evidence-based lifestyle interventions and pharmacologic strategies, including antithrombotic, antihypertensive, and lipid-lowering therapies, ACS patients remain at substantial risk for recurrent CV events.

A novel and clinically yet unexplored possible unmet medical need is the aggravation of atherosclerosis that has been observed in animal models after myocardial infarction and reperfusion. Recent observations have shown that this in part is explained by an increased reactivity of blood monocytes and bone marrow-derived macrophages to oxidized LDL. The circulating levels of oxidized and malondialdehyde-modified LDL are also known to be elevated in subjects with AMI. These observations suggest that targeting oxidized LDL may be of particular benefit in AMI and post-AMI patients.

### Description of Coronary Perivascular Inflammation Assessment Endpoints.

FAI (Fat Attenuation Index): an image biomarker that measures the attenuation gradients of the adipose tissue around human coronary arteries which is corrected for some basic technical scan parameters. FAI is an unadjusted value expressed in Hounsfield Units (HU).
FAI score: a standardized measurement of coronary inflammation for each coronary artery (adjusted for age, sex as well as technical, biological, and anatomical characteristics) to allow individualized interpretation of the degree of coronary inflammation.
CaRi-Heart Risk score: represents the individualized patient risk of a fatal cardiac event at 8 years which incorporates the FAI score values into a prognostic model that includes information about CCTA-derived plaque metrics and clinical cardiovascular risk factors (diabetes, smoking, hypertension, hyperlipidemia). FAI, FAI score, and CaRi-Heart risk score are determined using CaRi-Heart technology analysis performed on routine CCTA scans (CaRi-Heart, Caristo Diagnostics, Ltd., Oxford, United Kingdom).

### Investigational Plan

### Overall Study Design and Plan: Description

This multicenter, double-blind, randomized, placebo-controlled study is designed to provide confirmation of clinically meaningful bioactivity for orticumab in patients with a prior history of type 1 myocardial infarction who have quantifiable elevated coronary inflammation. A total of 240 randomized participants are planned, including 80 participants in each of the two active orticumab treatment groups and 40 participants in each of the two placebo treatment groups. The duration of study drug treatment is up to 24 weeks, including an initial IV infusion over 20 to 30 minutes followed by either monthly IV infusion or SC injections starting at Week 5.

Following the Screening Period (up to 45 days), confirmed eligible participants are randomized in a 2:2:1:1 ratio to:
1) Orticumab 1245 mg IV on Day 1, followed by 1245 mg IV every 4 weeks (6 IV doses in total),
2) Orticumab 1245 mg IV on Day 1, followed by 330 mg SC every 4 weeks for five monthly doses (1 IV dose and 5 SC doses in total),
3) Placebo IV on Day 1, followed by placebo IV every 4 weeks (6 IV doses in total), or
4) Placebo IV on Day 1, followed by placebo SC every 4 weeks for five monthly doses (1 IV dose and 5 SC doses in total).

Following completion of 24 weeks of treatment, participants have a 4-week follow-up visit after 28 weeks. Eligible participants undergo coronary CT angiography (CCTA) screening initially and at Month 6 (Week 24). Participants who discontinue from the study early have an exit CCTA whenever feasible. For the follow up scan at Week 24, if the scan is considered to be non-diagnostic for clinical evaluation by the site or the independent imaging core laboratory, the scan may be repeated one time. A full schedule of assessments is provided in Table 1.

### Study Visits

### Screening Visit/Period (Day-45 to Day-1)

Screened participants provide written informed consent and have a screening visit within 45 days of dosing. At the Screening visit, after written informed consent is obtained, the following study procedures are performed:
Collection of demographic data (sex, age, race and ethnicity)
Completion of medical and social history, including time of prior MI event, HIV and hepatitis status, tobacco, alcohol, and drug use, postmenopausal status (if applicable).
Review of prior and concomitant medications and supplements.
NYHA functional class is determined.
Physical examination is performed.
Height and weight are measured.
Measurement of vital signs (sitting or supine systolic and diastolic blood pressure, pulse rate after participant rests for at least 5 minutes)
Collection of blood following a fast for at least 8 hours for clinical safety laboratory tests, including hematology, chemistry, lipid panels and hemoglobin A1c.
Collection of blood for serum β-HCG, qualitative and quantitative assessment for women of childbearing potential.
Review of any baseline adverse events since first screened.
Review of eligibility criteria

Participants who are deemed potentially eligible undergo a CCTA to determine FAI score and CaRi-Heart risk score analysis (Caristo Diagnostics, Oxford UK) of their coronary arteries so as to evaluate the extent of elevated coronary inflammation. Participants who have a quantifiable Fat Attenuation Index (FAI) Score greater than or equal to the 50th percentile in at least two coronary arteries for their age group (50th percentile determined based on reference population data) or a quantifiable FAI Score greater than or equal to 75th percentile in at least one coronary artery for their age group (75th percentile determined based on reference population data) are eligible for randomization if all other eligibility criteria are also met. Baseline randomization stratification occurs as described.

Screening procedures may be done on separate days provided all required study procedures are completed within the 45 day time period and prior to randomization and study drug dosing. Participant eligibility for all screening study procedures (other than baseline CCTA) should be reviewed and confirmed by the investigator before the baseline CCTA is performed.
Baseline CCTA scan is performed.
Following completion of all Baseline assessments and the Baseline CCTA and confirmation of eligibility for randomization, the Baseline/Week 1 Day 1 Visit are scheduled. Participants are instructed to fast for at least 8 hours prior to the Baseline visit and to avoid alcohol for 72 hours prior to all study visits.
Follow up CCTA scan for Week 24 is scheduled for participants who are enrolled in the study.
Participants who do not meet eligibility criteria based on a nonqualified Baseline CCTA or failing other eligibility criteria are considered a screen failure and have the reason documented in their source records and in the database. Any baseline concomitant medications or adverse events are also recorded.

### Baseline/Week 1 Day 1 Visit (Visit 1)

Eligible participants have Baseline assessments including the following study procedures:
Weight is measured
Measurement of vital signs (sitting or supine systolic and diastolic blood pressure, pulse rate after participant rests for at least 5 minutes).
Collection of blood following a fast for at least 8 hours for the following predose laboratory tests:
   clinical safety laboratory tests, including hematology, chemistry, lipid panels
   cardiometabolic and inflammation biomarker laboratory tests
   ADA titer determination
Collection of blood sample for predose serum orticumab concentration determination
Collection of urine for pregnancy test for women of childbearing potential.
Resting 12-lead ECG is performed with the participant in the supine position after resting for at least 10 minutes
Concomitant medication use is reviewed and recorded.
Randomization is performed after the participant's eligibility is confirmed by the Investigator and after all Baseline study assessments are completed.

Baseline Randomization Stratification: Enrollment and randomization is stratified such that there is no more than 50% of randomized participants in the 50 to 75th centile for baseline FAI score in at least two coronary arteries for their age group. Participants are stratified into one of two groups, those in the 50 to 75th centile category and those in the >75th centile category.
After randomization, participants receive double-blind orticumab 1245 mg or matching placebo injectable solution (in 0.9% Sodium Chloride to make a total of 50 mL infusion) administered IV infusion over 20 to 30 minutes with close monitoring for any adverse events for at least 30 minutes following the end of the IV infusion. Vital signs (sitting or supine systolic and diastolic blood pressure and pulse rate) are measured approximately 30 minutes after the end of the IV infusion.
Study drug administration is documented.
Adverse events are monitored and recorded since the last visit and after study drug administration.
Upon completion of the Week 1 Day 1 visit, participants are instructed to return to the research site in the fasted state (for at least 8 hours) for double-blind monthly study drug dosing and other study procedures on Weeks 5, 9, 13, 17 and 21 (+3 day allowable time window for Weeks 5 through 21 visits).
   Weeks 5, 9, 13, 17 and 21 Visits (Visits 2 through 6)

Participants report to the research site on Weeks 5, 9, 13, 17 and 21 for the following study procedures:
Weight is measured
Measurement of vital signs (sitting or supine systolic and diastolic blood pressure, pulse rate after participant rests for at least 5 minutes)
   Collection of blood following a fast for at least 8 hours for the following predose laboratory tests: clinical safety laboratory tests, including hematology, chemistry
   lipid panels (Only at Week 13, Visit 4)
   cardiometabolic and inflammation biomarker laboratory tests
   ADA titer determination
Collection of blood sample for predose serum orticumab concentration determination
Concomitant medication use is reviewed and recorded.
Participants randomized to an IV treatment arm receive double-blind orticumab 1245 mg or matching placebo injectable solution (in 0.9% Sodium Chloride to make a total of 50 mL infusion) administered IV infusion over 20 to 30 minutes with close monitoring for any adverse events for at least 30 minutes after the end of the infusion. Vital signs (sitting or supine systolic and diastolic blood pressure and pulse rate) are measured approximately 30 minutes after infusion.
Participants randomized to a SC treatment arm receive orticumab 330 mg or matching placebo SC (two SC injections per dose), one in the right and one in the left side of the abdomen. Participants are monitored for any adverse events for at least 30 minutes after the SC injections. Vital signs (sitting or supine systolic and diastolic blood pressure and pulse rate) are measured approximately 30 minutes after the SC injections.
Study drug administration is documented.
Adverse events are reviewed and recorded since the last visit and after study drug administration.
Upon completion of each visit, participants are instructed to return to the research site in the fasted state (for at least 8 hours) for double-blind monthly study drug dosing.

### Week 24 Visit (Visit 7).

At this visit, the Month 6 (Week 24) CCTA scan is performed. If the scan is considered to be non diagnostic for clinical evaluation, the scan may be repeated one time.
Upon completion of all Week 24 study procedures, participants have a final Week 28 end of study visit scheduled.

### Week 28 Follow-Up Visit

Eligible participants have Baseline assessments including the following study procedures:
Weight is measured
Measurement of vital signs (sitting or supine systolic and diastolic blood pressure, pulse rate after participant rests for at least 5 minutes)
   Collection of blood following a fast for at least 8 hours for the following predose laboratory tests: clinical safety laboratory tests, including hematology, chemistry, lipid panels and hemoglobin A1c
   cardiometabolic and inflammation biomarker laboratory tests
   ADA titer determination
Collection of blood sample for final serum orticumab concentration determination.
Collection of urine for pregnancy test for women of childbearing potential.
Resting 12-lead ECG is performed with the participant in the supine position after resting for at least 10 minutes
Concomitant medication use is reviewed and recorded, including any medications administered during or after the Week 24 CCTA procedure and since the Week 21 visit.
Adverse events are monitored and recorded, including any during or after the Week 24 CCTA procedure and since the Week 21 visit.
Upon completion of all study procedures, participants are discharged from the study.

### End of Study Definition

The end of the study (“study completion”) is defined as the date of the last protocol-specified visit or assessment (including telephone contact) for the last participant in the study.

### Study Schematic and Schedule of Assessments

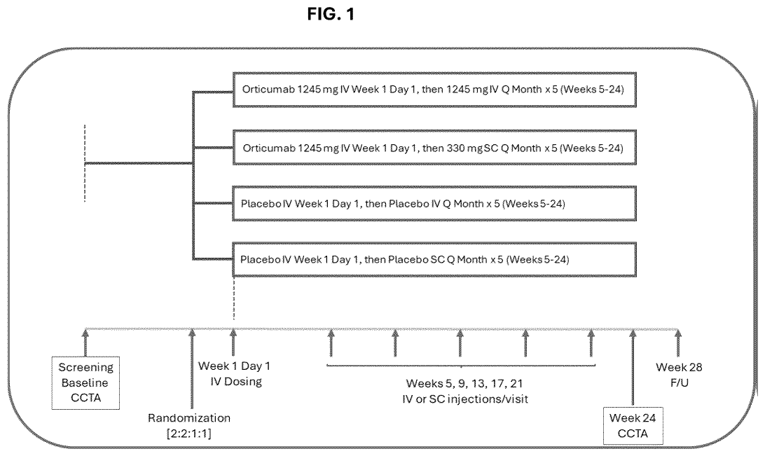

FIG. 1 presents a study schematic and Table 1 presents a schedule of assessments.

TABLE 1
Schedule of Assessments

| | Month | | | | | | | | | |
|---|---|---|---|---|---|---|---|---|---|---|
| | | Month | Month | Month | Month | Month | Month | | Month | |
| | | 1 | 2 | 3 | 4 | 5 | 6 | | 7 | |
| | Visit | | | | | | | | | |
| | | Visit | Visit | Visit | Visit | Visit | Visit | Visit | | |
| | Screening | 1 | 2 | 3 | 4 | 5 | 6 | 7 | F/U | E/T |
| | Day / Week | | | | | | | | | |
| | Day −45 to | Day 1 | Week | Week | Week | Week | Week | Week | Week | |
| | Day −1 | Week 1 | 5 | 9 | 13 | 17 | 21 | 24 | 28 | |
| Informed Consent | X | | | | | | | | | |
| Medical History / Medication | X | | | | | | | | | |
| History / Demographics | | | | | | | | | | |
| Eligibility Assessment | X | | | | | | | | | |
| Height and Weight | X | | | | | | | | | |
| NYHA assessment³ | X | | | | | | | | X | X |
| Physical Examination | X | | | | | | | | X | X |
| Vital Signs (BP and pulse rate)⁴ | X | X | X | X | X | X | X | | X | X |
| Clinical Safety Lab [symbol] | X | X | X | X | X | X | X | | X | X |
| Cardiometabolic and Inflammation | | X | X | X | X | X | X | | X | X |
| Biomarker Labs [symbol] | | | | | | | | | | |
| Serology [symbol] | X | | | | | | | | | |
| Pregnancy Test (females) [symbol] | X | X | | | | | | X | X | X |
| Serum LH & FSH (females) if | X | | | | | | | | | |
| needed to confirm postmenopausal | | | | | | | | | | |
| status [symbol] | | | | | | | | | | |
| 12-Lead Resting ECG [symbol] | | X | | | | | | | X | X |
| Randomization [symbol] | | X | | | | | | | | |
| Cardiac CT Angiography (CCTA)² | X | | | | | | | X | | X |
| Study Drug Administration [symbol] | | X | X | X | X | X | X | | | |
| Blood PK sampling for Serum | | X | X | X | X | X | X | | X | X |
| [symbol] Concentrations [symbol] | | | | | | | | | | |
| ADA Titer Sampling [symbol] | | X | X | X | X | X | X | | X | X |
| Body Weight | | X | X | X | X | X | X | | X | X |
| Price and Co [symbol] | X | X | X | X | X | X | X | | X | X |
| Medication Use | | | | | | | | | | |
| Adverse Event [symbol] | X | X | X | X | X | X | X | | X | X |
| Discharge from Study | | | | | | | | | X | X |

[symbol]

indicates data missing or illegible when filed

### Abbreviations and Definitions:

ADA=anti-drug antibodies; CT=computed tomography; CCTA=cardiac CT angiography; ECG=electrocardiogram; E/T=early termination; F/U=follow=up; IV=intravenous; PK=pharmacokinetic; SC=subcutaneous

### Schedule of Assessments Footnotes:

1 Double-Blind treatment dosing occurs on Week 1 Day 1, Week 5, Week 9, Week 13, Week 17, and Week 21 (±3 day window for Weeks 5 through 21 visits). The final Month 6 CCTA scan is performed at the Week 24 visit.
2 CCTA scans are performed during the Screening Period (Baseline scan) and a final CCTA scan is performed at Month 6 (Week 24). If a participant discontinues early from the study, an end of study CCTA scan is performed at the earliest feasible date following the last study treatment.
3 NYHA Functional Classification assessment classification is performed at Screening and at Week 28 or E/T visit if applicable.
4 Vital signs include sitting or supine systolic blood pressure, diastolic blood pressure and pulse rate measured prior to dosing. Blood pressure and pulse rate measurements should be obtained after a participant rests for at least 5 minutes.
5 Clinical laboratory tests including fasting chemistry panel, hematology lab tests are performed at Screening and predose at Baseline (Week 1 Day 1) and at Weeks 5, 9, 13, 17, 21, 28 and E/T if applicable. Lipid panel is performed at Screening and predose at Baseline (Week 1 Day 1), Visit 4 (Week 13) and F/U visit (Week 28). Hemoglobin A1c is performed at Screening and F/U visit (Week 28) only. Insulin is performed at Baseline (Week 1 Day 1) and F/U visit (Week 28) only. A clinical safety laboratory manual provides detailed instructions for sample collection and processing.
Hematology panel includes: Hematocrit, hemoglobin, red blood cell (RBC) count, white blood cell (WBC) count, differentials (neutrophils, eosinophils, basophils, lymphocytes, and monocytes), platelet count Chemistry panel includes: Albumin, alkaline phosphatase, alanine aminotransferase (ALT), aspartate aminotransferase (AST), blood urea nitrogen (BUN), calcium, chloride, carbon dioxide (CO2), creatinine, direct bilirubin, gamma-glutamyl-transferase (GGT), glucose, lactate dehydrogenase (LDH), phosphorus, potassium, sodium, total bilirubin, total cholesterol, total protein, and uric acid
Lipid panel includes fasting cholesterol, HDL; cholesterol, LDL (Friedwald equation); cholesterol, LDL (direct); cholesterol, Non-HDL, cholesterol, VLDL, and triglycerides
6 Cardiometabolic and inflammatory biomarkers are obtained in the fasting state (overnight 10 hour fast) predose at Baseline (Week 1), at Weeks 5, 9, 13, 17, 21 and 28, and E/T if applicable.
Cardiometabolic biomarkers and Inflammation biomarkers may include but not be limited to insulin and high-sensitivity C-reactive protein (hs-CRP). Additional samples (serum and plasma) are drawn and stored for possible additional cardiometabolic and/or inflammatory biomarkers. A cardiometabolic and inflammation biomarker laboratory manual provides detailed instructions for sample collection and processing.
7 Serology test for hepatitis C core antibody is performed only at Screening.
8 Pregnancy test (serum β-HCG, qualitative and quantitative at Screening, urine or serum pregnancy test at other visits when required) is performed on all female participants of childbearing potential at Screening, on Week 1 Day 1 prior to first study drug administration, on Week 24 prior to the Month 6 CCTA scan, and at Follow-Up Week 28 visit and E/T if applicable.
9 Serum FSH tests may be performed for female participants if required to confirm postmenopausal status at investigator discretion; postmenopausal status is documented by the Investigator based on medical history (and LH and FSH tests if obtained).
10 12-lead resting ECG is performed at Visit 1 (Week 1 Day 1) prior to study medication and at Follow-Up Week 28 visit and E/T if applicable. The ECG is performed in the supine position and should be done after the participant has rested for at least 10 minutes.
11 Randomization is be performed on Visit 1 (Week 1 Day 1) after the participant's eligibility is confirmed by the Investigator and after all Baseline study assessments are completed.
12 For the Week 1 Day 1 dose, all participants receive IV study drug consisting of double-blind orticumab 1245 mg or matching placebo (in 0.9% Sodium Chloride to make a total of 50 mL infusion) administered IV infusion over 20 to 30 minutes. For the monthly doses (Weeks 5 through 21 visits), participants randomized to an IV treatment arm receive orticumab 1245 mg or matching placebo (in 0.9% Sodium Chloride to make a total of 50 mL infusion) IV infusion over 20 to 30 minutes, and participants randomized to a SC treatment arm receive orticumab 330 mg or matching placebo SC (two SC injections per dose). Detailed instructions for preparing and administering the IV and SC doses are included in a pharmacy manual. Participants are monitored closely for adverse events for at least 30 minutes after the end of the IV infusion or after the SC injections. Vital signs (sitting or supine systolic and diastolic blood pressure and pulse rate) are measured approximately 30 minutes after the end of the IV infusion (all doses) and approximately 30 minutes after the first SC injections dose.
13 Blood samples are obtained prior to doses at each visit to determine serum trough concentrations of orticumab. Blood samples immediately after the IV infusion has been administered are obtained in a subset of participants to determine serum peak concentrations of orticumab. A Pharmacokinetic sample collection manual provides detailed instructions for PK sample collection and processing. Post-dose blood samples may be obtained at the time of expected peak serum orticumab concentration or at other times in a subset of participants.
14 ADA titer sample collections are performed at Baseline (Week 1) prior to study drug administration, and at Weeks 5, 9, 13, 17, 21, and 28 and E/T if applicable.

### Study Subject Selection

### Number of Subjects

A total of 240 randomized participants are planned, including 80 participants in each of the two active orticumab treatment groups and 40 participants in each of the two placebo treatment groups (2:2:1:1). Participants who discontinue prematurely are not replaced.

### Inclusion Criteria

Participants meeting the following criteria are included in the study:
1. Participant must provide informed consent before any study specific activities are performed, must be able and willing to meet all requirements for randomization and must adhere to the schedules of activities.
2. Participant must be >180 days after presumed type-1 myocardial infarction (i.e., due to plaque rupture or erosion, either STEMI or NSTEMI) without subsequent unstable or severe angina (Canadian Cardiovascular Society Class 3 or 4) at the time of enrollment. Participants who have undergone PCI are allowed.
3. Participant must be on a stable cardiovascular treatment regimen consistent with local treatment guidelines for post-AMI patients (such as maximally tolerated statin and/or PCSK9 inhibitor medication for LDL reduction, antiplatelet medication, and hypertension treatment).
4. Participant must have an evaluable, pre-randomization coronary CTA with one of the following:
   A quantifiable Fat Attenuation Index (FAI) Score greater than or equal to the 50th percentile (per reference standard) for their age group in at least two coronary arteries or
   A quantifiable Fat Attenuation Index (FAI) Score greater than or equal to the 75th percentile (per reference standard) in at least one coronary artery
5. Participant must have body mass index (BMI)≤40 kg/m².
6. Adult male and female patients ≥18 years of age at the Screening Visit:

### Study Treatment

### Description of Investigational Drug

The study drug (orticumab) is manufactured, bulk packaged, and labeled under good manufacturing practices (GMP). The study drug is shipped to each study site by Sponsor or their designee.

Active: Orticumab is a fully human recombinant IgG1 monoclonal antibody (molecular weight, approximately 144 kDa) that is expressed in Chinese hamster ovary cells. Orticumab is provided as a sterile liquid for infusion and contains no preservatives. Each single-use, 2 mL vial is designed to contain 1.5 mL of orticumab without added preservatives. The Drug Product is formulated as 150 mg/ml orticumab in 20 mM Histidine Acetate, 150 mM Arginine Acetate, 0.02% Polysorbate 20, pH 5.5.

Placebo: The placebo formulation is identical to the drug product, minus the active ingredient. For further details, see the Investigator Brochure.

### Drug Packaging

Adequate supplies of medication are made available to each investigational site. The study drug is packaged into kits containing two (2) vials of either orticumab (150 mg/mL) or placebo.

### Drug Labeling

Each kit and vial is labeled with the following information:
Protocol number.
Name and address of the Sponsor, including main contact.
Orticumab 150 mg/ml or Placebo (1.5 mL/vial), dosage form, route of administration, strength, and volume.
Treatment number/Unique 6-digit kit number.
Batch/Lot number.
Directions for use.
Storage conditions.
The following statement is on the drug label “Caution: New Drug-Limited by Federal (or United States) Law to Investigational Use”.

### Drug Storage

Glass vials containing study drug must be stored at a refrigerated temperature (2 °C-8 °C) and away from light until used. Since stability studies are ongoing, the study drug has an assigned re-test date that is updated during the course of the clinical trial. The re-test date is available in the Interactive Response Technology (IRT) system.

### Dosage and Administration of Treatment

Participants are randomly allocated in a 2:2:1:1 ratio to receive:
1) Orticumab 1245 mg IV on Day 1, followed by 1245 mg IV every 4 weeks (6 IV doses in total),
2) Orticumab 1245 mg IV on Day 1, followed by 330 mg SC every 4 weeks for five monthly doses (1 IV dose and 5 SC doses in total),
3) Placebo IV on Day 1, followed by placebo IV every 4 weeks (6 IV doses in total), or
4) Placebo IV on Day 1, followed by placebo SC every 4 weeks for five monthly doses (1 IV dose and 5 SC doses in total) based on the Schedule of Assessments (Table 1).

Preparation of the 1245 mg IV dose requires that a 10 ml syringe and needle withdraw 8.3 mL from the contents of six (6) vials in three kits and is diluted in 0.9% sodium chloride to make a total of 50 mL infusion bag which is administered via IV infusion over 20 to 30 minutes. Consult the pharmacy manual for dose preparation and administration instructions.

Preparation of the 330 mg SC dose requires that a 1.1 mL volume be drawn into each of two syringes making 165 mg dose per syringe. The 330 mg SC dose entails administering two SC doses, one in each side of the abdomen.

### Study Procedures and Assessments

### Efficacy Assessments

### Coronary Perivascular Inflammation Assessments

Fat Attenuation Index (FAI), FAI score and CaRi-Heart Risk score is determined for the 3 coronary arteries (RCA, LAD and LCX) from the Baseline and Week 24 CCTA procedure.

### Coronary Artery Plaque Volume Assessments

Coronary artery plaque volume assessments obtained during the Baseline and Week 24 CCTA procedure includes determination of the following parameters:
Non-calcified plaque volume for total (whole heart), per-vessel, and per-lesion
Low-attenuation (<30 HU) plaque volume: total, per-vessel, and per-lesion
Total coronary atherosclerotic plaque burden
Total calcified plaque volume
Coronary artery calcium volume and Agatston scores in non-stented vessels
Percent atheroma volume for total, calcified and non-calcified plaque

### Pharmacokinetic Assessments

Blood samples are obtained prior to doses at each visit to determine serum trough concentrations of orticumab.

Samples of venous blood for serum orticumab concentration determination are collected in K2EDTA tubes. A Pharmacokinetic sample collection manual provides detailed instructions for PK sample collection, processing and storage at the research site and shipping instructions to the designated bioanalytical laboratory.

### Cardiometabolic and Inflammation Biomarkers

Cardiometabolic and inflammatory biomarkers are obtained in the fasting state (overnight 10 hour fast) predose at Baseline (Week 1), at Weeks 5, 9, 13, 17, 21 and 28, and at the Early Termination visit, if applicable.

Cardiometabolic biomarkers and Inflammation biomarkers may include but not be limited to insulin and high-sensitivity C-reactive protein (hs-CRP). Additional samples (serum and plasma) are drawn and stored for possible additional cardiometabolic and/or inflammatory biomarkers. A cardiometabolic and inflammation biomarker laboratory manual provides detailed instructions for sample collection and processing.

### Efficacy Analyses

### Primary Efficacy Analysis

The primary endpoint is the percentage change from baseline at 6 months in the mean FAI Score for the three coronary arteries LAD, RCA, and LCX. The primary analysis of the primary endpoint comes from an analysis of covariance (ANCOVA) model with terms for treatment and baseline FAI Score (covariate).

The percent change from baseline at 6 months in the mean FAI score for the 3 coronary arteries measured by CCTA is summarized and analyzed using an ANCOVA model that includes terms for treatment and baseline FAI score as a covariate.

### Secondary Efficacy Analysis

Secondary efficacy analyses come from an analysis of covariance (ANCOVA) model with terms for treatment and baseline FAI Score (covariate) and include:
Change from baseline for FAI, FAI score (mean absolute change and mean percent change) and FAI score centile for orticumab compared to placebo after 6 months of treatment in the following vessels:
   Greatest change in most inflamed vessel
   Greatest change in any vessel
   RCA only analysis
   LAD only analysis
   LCX only analysis.
Mean absolute change from baseline for mean FAI and mean FAI score, defined as the average of the analyzable vessels (with valid baseline FAI and post-baseline FAI) across the three main coronary arteries (RCA, LAD, LCX)
Change from baseline for CaRi-Heart risk score (mean absolute change and mean percent change) for orticumab compared to placebo after 6 months of treatment

### Exploratory Efficacy Analysis

The observed and change from baseline in coronary artery plaque volume by vessel and total plaque volume are summarized and analyzed using a mixed effects linear model that includes fixed effects for treatment and site with baseline value as a covariate at each post-baseline visit in which CCTA is performed. These exploratory analyses include:
Change from baseline in non-calcified coronary artery plaque volume for total (whole heart), per-vessel, and per-lesion compared to placebo
Change in low-attenuation (<30 HU) plaque volume: total, per-vessel, and per-lesion compared to placebo
Change in total calcified plaque volume compared to placebo
Change in coronary artery calcium volume and Agatston scores in non-stented vessels
Change in percent atheroma volume (PAV) for total, calcified and non-calcified plaque
Change in radiotranscriptomic biomarkers of coronary inflammation

### Pharmacokinetic Data Analysis

Trough serum orticumab concentration data are summarized by treatment and visit using mean, standard deviation, percent coefficient of variation, geometric mean, median, minimum and maximum values.

### Cardiometabolic and Inflammation Biomarkers Analysis

The observed and change from baseline for the cardiometabolic biomarkers and the inflammation biomarkers are summarized descriptively and displayed graphically by treatment, visit and parameter.

### NYHA Functional Classification Analysis

NYHA functional classification is summarized by categories at Screening and at Week 28 or Early Termination visit. Any clinically relevant shifts in functional class are presented.

### Example 3. Administration of Orticumab to Reduce the Risk of MACE in a Patient

Patients who had a prior ACS may be administered a therapeutically effective amount of orticumab to reduce the risk of a MACE in the patient. Patients may have had an ACS within the past 24 hours. Patients may have had an ACS one week to three years prior. Patients may have had an ACS from one week to two years prior to the administration of orticumab. Patients may have had an ACS from two weeks to one year prior.

Orticumab may be administered to the patient having been identified as having had an ACS.

Orticumab may be administered to the patient starting with a loading dose of 1245 mg delivered intravenously. Orticumab may be administered in a plurality of subsequent doses of orticumab in an amount of 330 mg/month. The subsequent doses of orticumab may be administered in two or more doses over at least 8 weeks, 12 weeks, or 16 weeks. The maintenance doses of orticumab may be administered to the patient intravenously or subcutaneously.

Orticumab may be administered to the patient starting with a loading dose of 1245 mg delivered intravenously. Orticumab may be administered in a plurality of subsequent doses of orticumab in an amount of 165 mg every 2 weeks. The subsequent doses of orticumab may be administered in two or more doses over at least 8 weeks, 10 weeks, or 12 weeks. The maintenance doses of orticumab may be administered to the patient intravenously or subcutaneously.

Orticumab may be administered to the patient starting with a loading dose of 1245 mg delivered intravenously. Orticumab may be administered in a plurality of subsequent doses of orticumab in an amount of 165 mg/week. The subsequent doses of orticumab may be administered in two or more doses over at least 8 weeks, 10 weeks, or 12 weeks. The maintenance doses of orticumab may be administered to the patient intravenously or subcutaneously.

Orticumab may be administered to the patient starting with a loading dose of 1245 mg delivered intravenously. Orticumab may be administered in a plurality of subsequent doses of orticumab in an amount of 82.5 mg/week. The subsequent doses of orticumab may be administered in two or more doses over at least 8 weeks, 10 weeks, or 12 weeks. The maintenance doses of orticumab may be administered to the patient intravenously or subcutaneously.

The risk of a MACE may be measured within the ensuing 30 days, 60 days, 90 days, five years, four years, three years, two years, or one year. A reduced risk of a MACE may be determined if the patient does not experience a MACE in the ensuing five years, four years, three years, two years, or one year.

### Example 4. Administration of Orticumab and Lipid-Lowering Therapy to Reduce the Risk of MACE in a Patient Who had a Prior ACS

Patients who had a prior ACS may be administered a therapeutically effective amount of orticumab to reduce the risk of a MACE in the patient. Patients may have had an ACS within the past 24 hours. Patients may have had an ACS one week to three years prior. Patients may have had an ACS from one week to two years prior to the administration of orticumab. Patients may have had an ACS from two weeks to one year prior

The patient is also administered a lipid-lowering therapy. The lipid-lowering therapy may include administration of a lipid-lowering agent to the patient. The lipid-lowering agent may be a statin, wherein the statin may be lovastatin, pravastatin, simvastatin, fluvastatin, atorvastatin, rosuvastatin, or pitavastatin, or a combination thereof. The lipid-lowering agent may be ezetimibe or NNC0385-0434A. The lipid-lowering agent may be an antibody, wherein the antibody may be evinacumab or a PCSK9 antibody, such as evolocumab or alirocumab. The lipid-lowering agent may be an siRNA, wherein the siRNA may be olpasiran or a PCSK9 siRNA, such as inclisiran. The lipid-lowering agent may be an antisense oligonucleotide, wherein the antisense oligonucleotide may be mipomersen, volanesorsen, pelacarsen, or olezarsen. The lipid-lowering agent may be a small molecule. The small molecule may be a fibrate, wherein the fibrate may be fenofibrate, bezafibrate, gemfibrozil, or pemafibrate. The small molecule may be an omega-3 fatty acid, wherein the omega-3 fatty acid may be docosahexaenoic acid (DHA), eicosapentaenoic acid (EPA), or docosapentaenoic acid (DPA). The small molecule may be a bile acid sequestrant, wherein the bile acid sequestrant may be cholestyramine, colesevelam, or colestipol. The small molecule may be bempedoic acid or lomitapide.

Orticumab may be administered to the subject while they receive treatment by a lipid-lowering therapy. Orticumab may be administered to the subject who has previously been treated by a lipid-lowering therapy.

Orticumab may be administered to the patient starting with a loading dose of 1245 mg delivered intravenously. Orticumab may be administered in a plurality of subsequent doses of orticumab in an amount of 330 mg/month. The subsequent doses of orticumab may be administered in two or more doses over at least 8 weeks, 12 weeks, or 16 weeks. The maintenance doses of orticumab may be administered to the patient intravenously or subcutaneously.

Orticumab may be administered to the patient starting with a loading dose of 1245 mg delivered intravenously. Orticumab may be administered in a plurality of subsequent doses of orticumab in an amount of 165 mg every 2 weeks. The subsequent doses of orticumab may be administered in two or more doses over at least 8 weeks, 10 weeks, or 12 weeks. The maintenance doses of orticumab may be administered to the patient intravenously or subcutaneously.

Orticumab may be administered to the patient starting with a loading dose of 1245 mg delivered intravenously. Orticumab may be administered in a plurality of subsequent doses of the orticumab in an amount of 82.5 mg/week. The subsequent doses of orticumab may be administered in two or more doses over at least 8 weeks, 10 weeks, or 12 weeks. The maintenance dose of orticumab may be administered to the patient intravenously or subcutaneously.

There may be a reduced risk of a MACE when comparing rates for a MACE in patients treated with the orticumab in combination with a lipid-lowering therapy to patients relying solely on a lipid-lowering agent. There may further be a reduction risk of MACE when comparing severity of MACEs (e.g., number of MACEs) during or after the treatment. The patient's risk of experiencing an adverse cardiac event may be measured within the ensuing 30 days, 60 days, 90 days, five years, four years, three years, two years, or one year.

1. A method for reducing the risk of a MACE in a subject, the method comprising administering to the subject orticumab using a phased dosing schedule comprising:
(i) a loading dose comprising 1245 mg of orticumab; and
(ii) a maintenance dose comprising 330 mg of orticumab, wherein the maintenance dose is administered to the subject monthly.

2. A method for reducing risk of a MACE in a subject, the method comprising administering to the subject orticumab using a phased dosing schedule comprising:
(i) a loading dose comprising 1245 mg of orticumab; and
(ii) a maintenance dose comprising 165 mg of orticumab, wherein the maintenance dose is administered to the subject every two weeks.

3. A method for reducing the risk of a MACE in a subject, the method comprising administering to the subject orticumab using a phased dosing schedule comprising
(i) a loading dose comprising 1245 mg of orticumab; and
(ii) a maintenance dose comprising 165 mg of orticumab, wherein the maintenance dose is administered to the subject weekly.

4. A method for reducing the risk of a MACE in a subject, the method comprising administering to the subject orticumab using a phased dosing schedule comprising
(i) a loading dose comprising 1245 mg of orticumab; and
(ii) a maintenance dose comprising 82.5 mg of orticumab, wherein the maintenance dose is administered to the subject weekly.

5. The method of any one of claims 1-4, wherein the loading dose is administered intravenously.

6. The method of any one of claims 1-5, wherein the maintenance dose is administered subcutaneously.

7. The method of claim 1, wherein the maintenance dose is administered 1 month after the loading dose is administered to the subject.

8. The method of claim 2, wherein the maintenance dose is administered 2 weeks after the loading dose is administered to the subject.

9. The method of claim 3 or claim 4, wherein the maintenance dose is administered 1 week after the loading dose is administered to the subject.

10. The method of any one of claims 1-9, wherein the subject is administered orticumab for at least 28 weeks.

11. The method of any one of claims 1-10, wherein the subject has had a prior acute coronary syndrome (ACS).

12. The method of claim 11, wherein the prior ACS comprises unstable angina (UA).

13. The method of claim 11, wherein the prior ACS comprises a myocardial infarction (MI).

14. The method of claim 13, wherein the MI is ST-segment elevation myocardial infarction (STEMI).

15. The method of claim 13, wherein MI is non-ST-segment elevation myocardial infarction (NSTEMI).

16. The method of any one of claims 13-15, wherein the MI has occurred at least 6 months prior to administration of orticumab.

17. The method of any one of claims 1-16, wherein the subject has an FAI Percentile of ≥70.

18. The method of any one of claims 1-16, wherein the subject has an FAI Percentile of ≥50 and <70.

19. The method of any one of claims 1-18, wherein the subject's relative risk of a MACE is reduced by at least 20%.

20. The method of claim 19, wherein the subject's relative risk of a MACE is reduced by at between 20% and 99%.

21. The method of any one of claims 1-20, wherein the MACE is an MI.

22. The method of any one of claims 1-20, wherein the MACE is a stroke.

23. The method of any one of claims 1-20, wherein the MACE is resuscitated cardiovascular death.

24. The method of any one of claims 1-23, wherein the method further comprises administering a lipid-lowering agent to the subject.

25. The method of any one of claims 1-23, wherein the subject was previously on a lipid-lowering agent.

26. The method of claim 24 or 25, wherein the lipid-lowering agent comprises an antibody, an siRNA, an antisense oligonucleotide, a small molecule, an omega-3 fatty acid, a bile acid sequestrant, or a peptide.

27. The method of claim 26, wherein the small molecule is a statin.

28. The method of claim 27, wherein the statin is lovastatin, pravastatin, simvastatin, fluvastatin, atorvastatin, rosuvastatin, or pitavastatin.

29. The method of claim 26, wherein the small molecule is ezetimibe.

30. The method of claim 26, wherein the small molecule is a fibrate.

31. The method of claim 30, wherein the fibrate is fenofibrate, bezafibrate, gemfibrozil, or pemafibrate.

32. The method of claim 26, wherein the lipid-lowering agent is a bile acid sequestrant.

33. The method of claim 32, wherein the bile acid sequestrant is cholestyramine, colesevelam or colestipol.

34. The method of claim 26, wherein the small molecule is bempedoic acid or lomitapide.

35. The method of claim 26, wherein the lipid-lowering agent is an omega-3 fatty acid.

36. The method of claim 35, wherein the omega-3 fatty acid is docosahexaenoic acid, eicosapentaenoic acid, or docosapentaenoic acid.

37. The method of claim 26, wherein the lipid-lowering agent is an antibody.

38. The method of claim 37, wherein the antibody is a PCSK9 antibody.

39. The method of claim 38, wherein the PCSK9 antibody is evolocumab or alirocumab.

40. The method of claim 37, wherein the antibody is evinacumab.

41. The method of claim 26, wherein the lipid-lowering agent is an siRNA.

42. The method of claim 41, wherein the siRNA is a PCSK9 siRNA.

43. The method of claim 41, wherein the PCSK9 siRNA is inclisiran.

44. The method of claim 41, wherein the siRNA is olpasiran.

45. The method of claim 26, wherein the lipid-lowering agent is an antisense oligonucleotide.

46. The method of claim 45, wherein the antisense oligonucleotide is mipomersen, volanesorsen, pelacarsen, or olezarsen.

47. The method of claim 26, wherein the peptide is NNC0385-0434A.
